(19) United States
(12) Patent Application Publication
(10) Pub. No.: US 2024/0345016 A1
(43) Pub. Date: Oct. 17, 2024

(54) IMPEDANCE-BASED LIQUID BIOPSY SYSTEM AND METHOD FOR DETECTING AND SCREENING CANCER

(71) Applicant: ASIMA HEALTH INC., Caledon (CA)

(72) Inventors: Shalini GUPTA, Toronto (CA); Puja PRASAD, New Delhi (IN); Tejal DUBE, New Delhi (IN); Shantanu CHOWDHURY, Delhi (IN); Meenakshi VERMA, New Delhi (IN); Ankita SINGH, New Delhi (IN)

(21) Appl. No.: 18/683,145

(22) PCT Filed: Jan. 29, 2024
(86) PCT No.: PCT/IB2024/050820

§ 371 Date: Feb. 12, 2024

(30) Foreign Application Priority Data
Jan. 30, 2023 (IN) 202311005850

(51) Int. Cl. G01N 27/327; C12Q 1/6886; G01N 27/02

(52) U.S. Cl. CPC G01N 27/3275; C12Q 1/6886; G01N 27/026

(57) ABSTRACT

The present disclosure provides impedance-based detection of nucleic acids for screening of cancer. The present disclosure is based on the use of impedance spectroscopy to detect nucleic acids in solution in the clinical concentration range, without using any labels, amplification steps, bioreceptors, surface modification or complex electrode geometries. More particularly, the present disclosure leverages the structural variations in nucleic acids of healthy and cancer patients, to classify samples as cancer-positive or cancer-negative purely based on their electrophysiochemical properties. The present disclosure also provides a simple rapid, label-free miniaturized biosensor for detection and screening of cancer.

### CROSS-REFERENCE TO RELATED APPLICATION

The present application claims priority to Indian patent application No. 202311005850 filed on Jan. 30, 2023, and titled “IMPEDANCE-BASED LIQUID BIOPSY SYSTEM AND METHOD FOR DETECTING AND SCREENING CANCER”, the contents of which is incorporated herein by reference in its entirety.

### SEQUENCE LISTING

This application contains a Sequence Listing submitted as an extensible markup language (xml) named “P12615PC00_Sequence_Listing_ST.26.xml”, having a size in bytes of 17 kb, and created on Jan. 18, 2024. The information contained in this electronic file is hereby incorporated by reference in its entirety.

### FIELD

The present disclosure relates generally to the field of cancer screening. More particularly, the present disclosure provides a novel approach for DNA detection using impedance spectroscopy for cancer screening.

### BACKGROUND

Liquid biopsy is a minimally invasive diagnostic approach that has emerged as a promising tool for cancer screening and management through evaluation of blood-based biomarkers. Circulating tumor cells (CTCs), genomic DNA (gDNA), circulating tumor DNA (ctDNA), and cell free DNA (cfDNA) are some of the most widely studied biomarkers under this approach. ctDNA are tumor-derived fragmented DNA that are released in blood from apoptotic or necrotic cancer cells. Their concentration levels thus increase in blood during cancer and can be used to track disease progression. The most common platforms for DNA detection are next-generation sequencing (NGS), digital PCR, real-time PCR, and mass spectrometry. All of these methods require highly complex and expensive infrastructure which limits their adoption in a clinical setting. They are also highly time-consuming as they do not have the requisite sensitivities to yield results without signal or target amplification. No single technique exists so far that can detect ctDNA in a simple and rapid fashion, directly at the low concentration it exists in blood, without an added amplification step.

In addition to sequencing, aberrant DNA methylation has also been widely studied as an indicator of cancer development and progression. Some of the common techniques for cancer detection based on methylation patterns in plasma DNA include methylated DNA immunoprecipitation-sequencing (MeDIP-seq), bisulfite sequencing and hydroxymethylcytosine sequencing. MeDIP-seq utilizes a 5mC (methylcytosine) specific antibody to capture non-specifically fragmented methylated DNA strands. This is followed by labeling of the unenriched DNA with different fluorescent dyes (e.g., Cy5 and Cy3) and their co-hybridization onto a microarray platform. The ratio of the fluorescence intensity of the two dyes reflects the methylation status of the region of interest. The main limitations of this approach are that it requires single-stranded DNA for analysis and the quality of the anti-5mC antibodies can also vary significantly. The bisulfite sequencing employs bisulfite treatment of DNA to deaminate cytosine into uracil, and the converted residues are then read as thymine. The challenge is that the bisulfite conversion can lead to DNA fragmentation resulting in the generation of chimeric products. Since circulating DNA is already short, further fragmentation during bisulfite treatment can be of concern, as it can potentially reduce the sensitivity of the assay.

Recent and comparatively faster methods for DNA detection include colorimetry, fluorescence, and impedance spectroscopy. DNA-electrochemical biosensors in particular have received great attention due to their robustness, case of miniaturization, low detection limits, and compatibility with biological fluids. Traditionally, DNA detection by integrated sensors requires the molecule to be immobilized on the electrode surface using a bio-affinity receptor followed by its signal amplification using a set of probes or chemically active compounds. This not only adds extra steps and cost of reagents to the process but also increases the chances of false positives due to non-specific adsorption of the molecules on the surface. The electrode design/material in turn needs to undergo extensive optimization for the various binding steps but the chips generally cannot be reused, making the process less eco-friendly and economically inefficient. Some recent studies have tried to address these issues by measuring the electrical properties of DNA directly in solution, however, their signal sensitivity is low and greatly influenced by the type of solvent used. In deionized (DI) water, the lower limit of DNA detection (LOD) was found to be >10 mg/ml (Ma et al., Scientific reports, 2013, 3, 2730).

Thus, the available methods suffer from one or more limitations and there is a need for development of improved systems and methods for label-free detection of DNA for cancer diagnosis and screening without using surface modification, tedious amplification steps, or complex electrode geometries.

The present disclosure provides a novel impedance approach for DNA detection and cancer stratification by leveraging the impact of DNAs' structural aberrations on their electrophysiochemical properties. The said approach involves the use of impedance spectroscopy to ensure direct, rapid, sensitive, and label-free detection of DNA for cancer screening.

### SUMMARY

It is an aspect of the disclosure to provide a biosensor for analyzing a test sample that may include cfDNA. The biosensor also includes a microchamber to hold said test sample suspended in a zwitterionic buffer, where said microchamber encloses a plurality of electrodes; and an impedance analyzer operably connected to said microchamber and a programmable controller along with a digital processing unit to receive information from the plurality of electrodes, where impedance signals are measured by placing said test sample on the plurality of electrodes.

The biosensor where the microchamber may include a material selected from polydimethylsiloxane, polylactic acid, polylactic-co-glycolic acid, polyether ether ketone, silicone, nitrile, polyurethane, soft vinyl chloride resin, polypropylene, polyamide, polyethylene, polycarbonate, acrylonitrile butadiene styrene (ABS) resin, polystyrene, and poly(methyl methacrylate).

The biosensor may be for use in rapid, label-free, and amplification-free screening of cancer.

It is a further aspect of the disclosure to provide a method for liquid biopsy. The method also includes: collecting a biological sample and extracting a cfDNA fraction from said biological sample; suspending said cfDNA fraction in the zwitterionic buffer to prepare a test sample; loading said test sample over a plurality of electrodes enclosed inside a microchamber; measuring the electrical conductivity of said test sample using an impedance analyzer; and analyzing a difference between the electrical conductivity of said test sample and the electrical conductivity of a control sample.

The method where the biological sample may include a biological fluid selected from whole blood, plasma, platelets, saliva, white blood cells, serum, and urine. In examples where the biological sample includes plasma, the method may include separating the plasma from whole blood via double centrifugation, the double centrifugation may include a first cycle at 1500 g to 2500 g and preferably 2000 g for 7-12 minutes and preferably 10 minutes, and a second cycle at 15000-17000 g and preferably 16000 g for 7-12 minutes and preferably 10 minutes at 4° C.

The zwitterionic buffer may be selected from (4-(2-hydroxyethyl)-1-piperazineethanesulfonic acid) (HEPES), piperazine-1,4-bis(2-hydroxypropanesulfonic acid), and 4-(2-hydroxyethyl)-1-piperazinepropanesulfonic acid.

The method can be used for quantitative and qualitative detection of cfDNA.

The method may have a limit of detection of 0.4 ng/ml or 5.14 pm and a sensitivity and specificity each of about 95% for cfDNA in HEPES buffer.

The method may have a detection time of less than 5 minutes and preferably less than 1 minute.

One general aspect includes use of impedance spectroscopy in the detection of cancer based on the electrical conductivity of a test sample that may include cfDNA in solution.

One general aspect includes a label-free method of detecting cfDNA using impedance spectroscopy. The label-free method also includes a. collecting a biological sample and extracting a cfDNA fraction from said biological sample; b. suspending said cfDNA fraction in a liquid to prepare a test sample, c. loading said test sample over a plurality of electrodes enclosed inside a microchamber, d. measuring the electrical conductivity of said test sample using an impedance analyzer, and e. analyzing a difference between the electrical conductivity of said test sample and the electrical conductivity of a control sample.

Implementations may include one or more of the following features. The method where the liquid is selected from a zwitterionic buffer, deionized water, and purified water. The biological sample may include a biological fluid selected from whole blood, plasma, platelets, saliva, white blood cells, serum, and urine. The biological sample may include plasma, the method may include separating the plasma from whole blood via double centrifugation, the double centrifugation may include a first cycle at 1500 g to 2500 g and preferably 2000 g for 7-12 minutes and preferably 10 minutes, and a second cycle at 15000-17000 g and preferably 16000 g for 7-12 minutes and preferably 10 minutes at 4° C. The buffer may include a zwitterionic buffer selected from (4-(2-hydroxyethyl)-1-piperazineethanesulfonic acid), piperazine-1,4-bis(2-hydroxypropanesulfonic acid), and 4-(2-hydroxyethyl)-1-piperazinepropanesulfonic acid. Said method can be used for quantitative and qualitative detection of cfDNA. Said method has a detection time less than 5 minutes and preferably less than 1 minute.

One general aspect includes a method of detecting DNA using impedance spectroscopy. The method also includes a. collecting a biological sample and extracting a nucleic acid fraction from said biological sample; b. suspending said nucleic acid fraction in a zwitterionic buffer to prepare a test sample, c. loading said test sample over a plurality of electrodes enclosed inside a microchamber, d. measuring the electrical conductivity of said test sample using an impedance analyzer, and e. analyzing a difference between the electrical conductivity of said test sample and the electrical conductivity of a control sample.

The nucleic acid fraction may include at least one of genomic DNA (gDNA), cell free DNA (cfDNA), mitochondrial DNA (mtDNA), and complementary DNA (cDNA).

The biological sample may include a biological fluid selected from whole blood, plasma, platelets, saliva, white blood cells, serum, and urine.

In embodiments where the biological sample includes whole blood, the method may include separating a buffy layer from whole blood. The zwitterionic buffer is selected from (4-(2-hydroxyethyl)-1-piperazineethanesulfonic acid), piperazine-1,4-bis(2-hydroxypropanesulfonic acid), and 4-(2-hydroxyethyl)-1-piperazinepropanesulfonic acid. Said method can be used for quantitative and qualitative detection of DNA.

The method may have a detection time of less than 5 minutes and preferably less than 1 minute.

Additional objects, advantages, and novel features of the disclosure will be set forth in part in the description which follows, and in part will become apparent to those skilled in the art upon examination of the following or may be learned by practice of the biosensor and methods described herein.

### BRIEF DESCRIPTION OF FIGURES

The accompanying figures illustrate some of the embodiments of the present disclosure together with the descriptions. These figures have been provided by way of illustration and not by way of limitation.

FIG. 1A is a schematic diagram representing the biosensor for impedance-based detection of DNA for cancer screening.

FIG. 1B is a schematic representing a method for impedance-based detection of DNA for cancer screening.

FIG. 2 is a graph depicting frequency optimization to differentiate healthy versus cancer cfDNA. The maximum change in signal was observed between 10 kHz to 1 MHz. Results are depicted for three healthy and three cancer samples having the following concentrations, respectively: 40.92 ng/ml, 54.18 ng/ml, 58.31 ng/ml, 79.92 ng/ml, 89.54 ng/ml, and 99.90 ng/ml.

FIG. 3 shows the effect of different media on the impedance response of cancer and healthy plasma samples (cfDNA concentration 53.50 ng/ml). Data are reported as the mean of three replicates ±2 SD. The samples prepared in Milli-Q™ and DI water are depicted on the left-Y axis, while the rest of the samples prepared in buffer solutions are shown on the right-Y axis.

FIG. 4A is a box and whisker plot of cfDNA concentrations in healthy and cancer clinical plasma samples. The upper border of the box indicates the upper quartile (75^{th} percentile) while the lower border indicates the lower quartile (25^{th} percentile), and the horizontal line in the box is the median.

FIG. 4B is a graph showing a comparative analysis of impedance signals in 15 mM HEPES pH 7.4 containing elution buffer.

FIG. 4C is a graph showing the number of samples for types of cancers investigated.

FIG. 4D is a graph showing an ROC analysis of the clinical samples.

FIG. 5 shows the effect of spiked cfDNA concentration on the impedance signal.

The data are reported as the mean of two replicates ±1 SD.

FIG. 6 illustrates representative TEM micrographs of healthy and cancer cfDNA suspended in 15 mM HEPES buffer pH 7.4. The images illustrate clear morphological differences between the two cfDNA types at varying concentrations.

FIG. 7A is a graph showing the effect of methylation on the impedance signal of single stranded (ss) and double stranded (ds) synthetic oligonucleotides.

FIG. 7B is a series of TEM images of 25% methylated and 100% methylated ds-oligonucleotides taken at 70 ng/ml and 140 ng/ml concentrations.

FIG. 8A is a graph of impedance as a function of oligo concentration for various sequences.

FIG. 8B is a chart of the sequences tested in FIG. 8A.

### DETAILED DESCRIPTION

At the very outset, it may be understood that the ensuing description only illustrates a particular form of this invention. However, such a particular form is only an exemplary embodiment, without intending to imply any limitation on the scope of this disclosure. Accordingly, the description and examples are to be understood as exemplary embodiments for teaching the disclosure and not intended to be taken restrictively.

The details of one or more embodiments of the disclosure are set forth in the accompanying description below including specific details of the best mode contemplated by the inventors for carrying out the invention, by way of example. It will be apparent to one skilled in the art that the present disclosure may be practiced without limitation to these specific details.

### ABBREVIATIONS USED

cfDNA Cell free DNA
cDNA Complementary DNA
ctDNA Circulating tumor DNA
EDTA Ethylene diamine tetra acetate
EPPS 4-(2-Hydroxyethyl)-1-piperazinepropanesulfonic acid
gDNA genomic DNA
gRNA guide RNA
HEPES (4-(2-hydroxyethyl)-1-piperazineethanesulfonic acid)
IDEs Interdigitated electrodes
LOD Limit of Detection
M Molar
mg Milligram
min Minutes
ml Milliliter
mM Millimolar
mtDNA Mitochondrial DNA
μg Microgram
μl Microlitre
μm Micrometer
μM Micromolar
nm Nanometer
PBMC Peripheral blood mononuclear cell
PBS Phosphate Buffered Saline
PDMS Polydimethylsiloxane
PIPES Piperazine-N,N′-bis(2-ethanesulfonic acid
PLA Polylactic acid
PLGA Polylactic-co-glycolic acid
PMMA Poly(methyl methacrylate)
PNA Peptide nucleic acid
POPSO Piperazine-1,4-bis(2-hydroxypropanesulfonic Acid)
ROC Receiver Operating Characteristic
S/N Signal to noise ratio
TEM Transmission electron microscopy
UV Ultraviolet
Z Impedance

### Definitions

The use of “comprise”, “comprises”, “comprising”, “contain”, “contains”, “containing”, “include”, “includes”, and “including” are not intended to be limiting. It is to be understood that both the foregoing general description and this detailed description are exemplary and explanatory only and are not restrictive.

As used herein, biotechnological terms have their conventional meaning as illustrated by the following illustrative definitions.

The term “Impedance” used herein refers to the effective resistance of an electric current or component to an altering current arising from combined effects of ohmic resistance and reactance.

The term “Biomarker” used herein refers to the biological marker which is a measurable indicator of some biological state or condition.

“Zwitterionic” is used herein to describe a molecule that possesses both a positive and a negative electrical charge. The terms “zwitterion”, “inner salt” or “dipolar ion” may be used interchangeably herein to describe a zwitterionic molecule.

Unless otherwise defined, scientific and technical terms used herein shall have the meanings that are commonly understood by those of ordinary skill in the art. Further, unless otherwise required by context, singular terms shall include pluralities and plural terms shall include the singular.

The foregoing broadly outlines the features and technical advantages of the present disclosure in order that the detailed description of the disclosure that follows may be better understood. It should be appreciated by those skilled in the art that the conception and specific embodiment disclosed may be readily utilized as a basis for modifying the disclosed methods or for carrying out the same purposes of the present disclosure.

The present disclosure provides a novel approach for nucleic acid detection in solution based on the effect of structural aberrations in the nucleic acid on the electrophysiochemical properties of nucleic acid to stratify cancer and healthy samples using impedance spectroscopy. In some embodiments, the present disclosure employs zwitterionic buffers having an intrinsically low conductivity at physiological pH in combination with impedance spectroscopy. The structure and low ionic conductivity of said buffers in the background allows measuring even the smallest electrical conductivity differences between the nucleic acid molecules of healthy individuals and cancer patients. These differences likely result from the differences in the solvation properties of the cancer and healthy nucleic acids because of structural variations in their methylation patterns. These structural dissimilarities can vary significantly in genomic DNA and accordingly in shorter DNA molecules such as cfDNA, giving rise to differences in their dielectric properties.

Aberrant methylation is a hallmark of oncogenesis, and differences in methylation patterns between tumors and benign tissues have been reported in many cancer types. A characteristic feature of epigenetic remodelling in cancer gDNA is the differential hypomethylation in the coding and intergenic regions and differential hypermethylation in the CpG-rich regulatory regions such that the overall genomic landscape is markedly hypomethylated. cfDNA fragments derived from gDNA are more selectively enriched in coding and intergenic regions compared to the gene promoter regions in both malignant and benign samples. This implies that the structural variations in cancer and healthy cfDNA described in the examples herein arise from the differences in their methylation patterns, with healthy cfDNA containing a much higher number of methyl groups. It is the presence of these non-polar functional methyl groups that leads to the hydrophobic self-assembly of healthy cfDNA in aqueous solution. Since the loss of methyl groups from the cytosines is gradual, the morphology changes from microaggregates to dispersed nanoaggregates as the cancer progresses. More importantly, since the global loss in methylation is a common signature shared by multiple cancer types, the electrophysicochemical properties and impedance signals also behave similarly across different cancer types.

The present disclosure provides highly sensitive impedance readings with high signal to noise ratio (S/N) and thus, the present impedance spectroscopy-based approach can be used for the direct, rapid, and label-free detection of DNA for cancer screening and diagnosis.

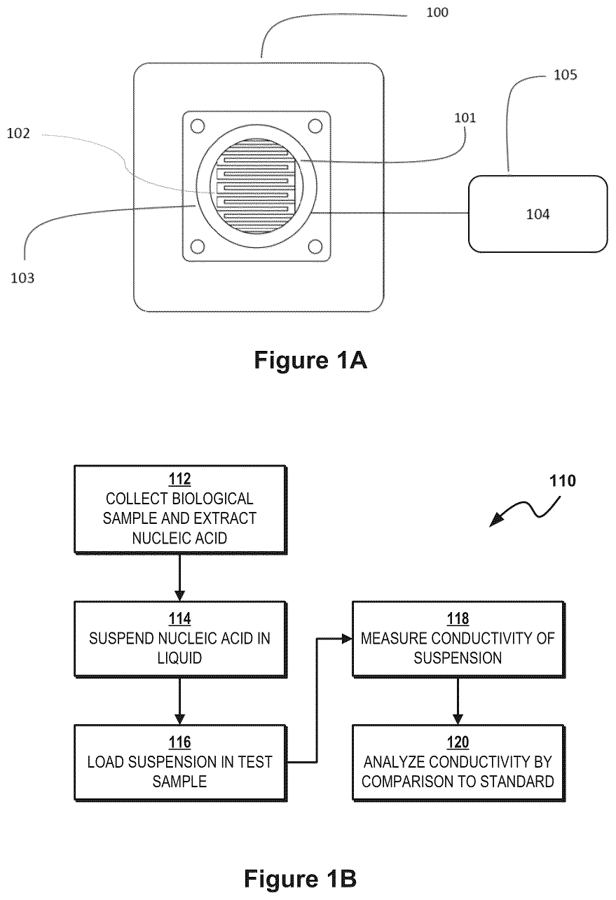

In an embodiment, the present disclosure provides a biosensor (100) for analyzing a test sample comprising: a microchamber (101) configured to hold said test sample suspended in a zwitterionic buffer, wherein said microchamber encloses a plurality of electrodes (102); and an impedance analyzer (105) operably connected to said microchamber and a programmable controller along with a digital processing unit (104) to receive information from the interdigitated microelectrodes; wherein the impedance signals are measured by placing said test sample on the plurality of electrodes enclosed inside the microchamber. FIG. 1A provides a schematic representation of the biosensor for impedance-based detection of cfDNA using a zwitterionic buffer.

The electrodes (102) may comprise any suitable type of electrode including, but not limited to, interdigitated micro electrodes (IDEs), microdisk electrodes, microband electrodes, and a three-dimensional microelectrode array.

The microchamber (101) may comprise an opening for receiving the test sample. The opening of the microchamber (101) may be covered with a seal (103). Any suitable material may comprise the seal (103) including but not limited to glass, film, pressure-sensitive adhesive tapes, polymers, and the like. In specific non-limiting examples, the seal (103) comprises a glass coverslip, Parafilm™, Scotch™ Tape, or a transparency slide. In some examples, the seal (103) is removably attachable to the microchamber (101). In further examples, the seal (103) rests on the microchamber (101). The seal (103) may reduce evaporation and contamination of the test sample.

The microchamber in the biosensor may comprise a material selected from but not limited to polydimethylsiloxane (PDMS), polylactic acid (PLA), polylactic-co-glycolic acid (PLGA), polyether ether ketone, silicone, nitrile, polyurethane, soft vinyl chloride resin, polypropylene, polyamide, polyethylene, polycarbonate, acrylonitrile butadiene styrene (ABS) resin, polystyrene, and poly(methyl methacrylate) (PMMA).

FIG. 1B is a schematic diagram showing a method (110) of impedance spectroscopy. In the examples described herein, the method (110) is performed by the biosensor (100). The method comprises the steps of:
a. collecting a biological sample and extracting a nucleic acid fraction from said sample (112);
b. suspending said nucleic acid fraction in a liquid to prepare the test sample (114);
c. loading said test sample over a plurality of electrodes enclosed inside a microchamber as defined in claim 1 (116);
d. measuring the electrical conductivity of said test sample using an impedance analyzer (118); and
e. analyzing the differences in electrical conductivity of said test sample by comparison with the standard (120).

Block 112 comprises collecting a biological sample and extracting a nucleic acid fraction from said sample. The biological sample may comprise a biological fluid selected from but not limited to whole blood, plasma, platelets, saliva, white blood cells, serum, urine, saliva, cerebrospinal fluid, amniotic fluid, bone marrow, and synovial fluid.

In embodiments where the biological sample comprises plasma, plasma may be obtained by separating a blood sample. In particular examples, the plasma is separated from blood via double centrifugation at 1500 g to 2500 g preferably 2000 g for about 7-12 mins preferably 10 min followed by 15000-17000 g preferably 16000 g for about 7-12 mins preferably 10 mins at about 4° C.

The nucleic acid fraction may comprise any suitable type of DNA, including but not limited to, genomic DNA (gDNA), cell free DNA (cfDNA), mitochondrial DNA (mtDNA), complementary DNA (cDNA), guide RNA (gRNA) and combinations thereof.

Any suitable means known in the art may be used to extract the nucleic acid fraction from the biological sample, including but not limited to phenol-chloroform, cetyltrimethylammonium bromide, silica columns, anion exchange columns, Chelex™ resin, salting out, solid-phase reversible immobilisation (SPRI), and combinations thereof. The method of nucleic acid extraction may be selected to extract a subset of the total nucleic acids in the biological sample.

In specific examples where the nucleic acid fraction comprises cfDNA, the cfDNA may be extracted with a MagMax™ Cell-Free DNA Isolation Kit (Thermo Fisher Scientific; Waltham, Massachusetts).

In specific examples where the nucleic acid fraction comprises guide RNA or genomic DNA, the extraction may include centrifuging whole blood at 2000 g for 10 minutes at 4° C., resulting in 3 layers: plasma (top), buffy coat (middle), and red blood cells (bottom). In specific examples where the nucleic acid fraction comprises gRNA, the buffy coat layer may be collected and mixed with a TRIzol® reagent (Invitrogen, Cat. No. 15596026). The RNA pellet may be dissolved in RNAase-free water and quantitated within 2 hours of isolation by UV-Visible spectroscopy. The extract may be used directly for analysis or stored at −80° C. In specific examples where the nucleic acids comprise gDNA, the gDNA may be extracted from the buffy coat layer with a KingFisher™ Blood DNA Kit (Thermo Fisher Scientific, Cat. No. 97010196) or any other standard method.

In specific examples where the nucleic acid fraction comprises mtDNA, the mtDNA may be extracted by isolating the PBMCs (Peripheral blood mononuclear cells) from whole blood using Ficoll-Hypaque density gradient centrifugation. Briefly, blood is layered on ficoll-paque and spun at 1800 rpm for 30 minutes. This results in 4 layers: plasma, buffy coat (with PBMCs), ficoll, and red blood cells (RBCs). The white buffy coat layer is collected and mixed with PBS for a total volume of 45 ml. The mixture is centrifuged at 1300 rpm for 7 minutes to pellet the cells. The cells are washed with PBS. The mtDNA is isolated from the PBMCs using a Mitochondrial DNA Isolation Kit (Abcam, ab 65321).

In some examples, block 112 further includes amplifying the nucleic acids. Any suitable method of amplification may be used including but not limited to polymerase chain reaction (PCR), isothermal amplification, multiple displacement amplification, and ligase chain reaction.

Block 114 comprises suspending said nucleic acid fraction in a liquid to prepare the test sample. The volume of liquid may be selected such that the concentration of nucleic acids in the test sample is equal or approximately equal to the concentration of the nucleic acid fraction in the biological sample. In other examples, the volume is selected such that the concentration of nucleic acids in the test sample is equal or approximately equal to a pre-determined standard. As part of block 114 process, the concentration of nucleic acids in the nucleic acid fraction may be quantified. The method of quantifying the nucleic acids is not particularly limited. In specific examples, the nucleic acids may be quantified with UV-Visible spectroscopy (Nanodrop™ Lite, Thermo Fisher Scientific).

The liquid in which the nucleic acid fraction is suspended may include but is not limited to, water, a zwitterionic buffer, and combinations thereof. In particular examples, where the liquid is water, the liquid may comprise ultrapure water, Milli-Q™ water (MilliporeSigma; Burlington Massachusetts), deionized water, or the like.

In examples where the liquid comprises a zwitterionic buffer, the zwitterionic buffer may comprise a Good's buffer. Suitable examples of Good's buffers include but are not limited to (4-(2-hydroxyethyl)-1-piperazinecthanesulfonic acid) (HEPES), piperazine-1,4-bis(2-hydroxypropanesulfonic acid) (POPSO) and 4-(2-Hydroxyethyl)-1-piperazinepropanesulfonic acid (EPPS).

The concentration of the zwitterionic buffer may be between about 1 mM and about 100 mM. In specific examples, the concentration of the zwitterionic buffer is about 5 mM. In specific examples, the concentration of the zwitterionic buffer is about 10 mM. In further examples, the concentration of the zwitterionic buffer is about 15 mM. In yet further examples, the concentration of the zwitterionic buffer is about 20 mM.

Block 114 may further comprise adjusting the pH of the liquid. In specific non-limiting examples, the pH of the liquid is adjusted to about 7.4.

Block 116 comprises loading said test sample over the plurality of electrodes (102) in the biosensor (100). Block 116 may comprise adding the test sample into the microchamber such that the test sample covers at least two of the plurality of electrodes (102). After loading the test sample into the microchamber, the microchamber (101) may be enclosed with the seal (103) to prevent evaporation of the test sample.

Block 118 comprises measuring the electrical conductivity of said test sample using an impedance analyzer. As part of block 118, the impedance analyzer (105) applies a test signal to one of the electrodes (102). The frequency of the test signal may be between about 1 Hz to about 1 MHz range. In particular examples, the frequency of the test signal may be about 0.1 MHZ. Generally, the frequency of the test signal is selected to maximize the difference between measurements for cancerous and healthy samples. As part of block 118, the impedance analyzer (105) measures the response signal in one of the electrodes. To calculate the electrical conductivity of the test sample, the impedance analyzer (105) compares the test signal to the response signal. In some examples, the test signal is applied to a first one of the electrodes and the response signal is measured in a second one of the electrodes. In other examples, the test signal is applied to the same electrode in which the response signal is measured.

Block 120 comprises analyzing the differences in electrical conductivity of the test sample by comparison with a standard. In biosensor (100), block 120 may be performed by the impedance analyzer (105) or a processor connected to the impedance analyzer. The standard may be retrieved from memory at the impedance analyzer or the processor. In some examples, the standard is obtained by measuring the electrical conductivity of the liquid, in which no nucleic acids are suspended. In other examples, the standard is obtained by measuring the electrical conductivity of synthetic oligonucleotides at a known concentration. In some examples, the standard is obtained by measuring the electrical conductivity of nucleic acids from a subject that is known to be non-cancerous or healthy. In yet further examples, the standard is obtained by measuring the electrical conductivity of nucleic acids from a subject that is known to be cancerous. Generally, the standard should be measured using the same method and the same or identical biosensor that was used to measure the electrical conductivity of the test sample. The dimensions of the microchamber and other variables may affect the electrical conductivity determined through this method (110).

The standard may comprise a single value or a range of values. In some examples, block 120 comprises comparing the electrical conductivity of the test sample to a plurality of standards.

Based on the comparison to a standard, block 120 may further include determining whether or not the biological sample is cancerous. The determination may be output at a display connected to the impedance analyzer (105) or a processor.

In some examples, the standard comprises a pre-determined threshold. If the electrical conductivity of the test sample is above the pre-determined threshold, the test sample is determined to be cancerous. If the electrical conductivity of the test sample is below the pre-determined threshold, the test sample is determined to be non-cancerous (healthy).

In some examples, block 120 includes determining whether the electrical conductivity of the test sample is closer to the electrical conductivity of a non-cancerous (healthy) standard or a cancerous standard. If the electrical conductivity is closer in value to the non-cancerous standard, the biological sample is determined to be non-cancerous. If the electrical conductivity is closer in value to the cancerous standard, the biological sample is determined to be cancerous.

In examples where the standard comprises a range of values, the determination at block 120 may include determining whether the electrical conductivity of the test sample falls within the range or not. If the electrical conductivity of the test sample falls within the range of a cancerous standard, the biological sample is determined to be cancerous. If the electrical conductivity of the test sample falls within the range of a non-cancerous standard, the biological sample is determined to be healthy.

Although block 120 has been described with respect to cancerous and non-cancerous standards, it should be understood that block 120 can similarly determine qualitative traits of the biological sample, such as the type or progress of cancer. In some examples, the standard represents a sample of nucleic acid obtained from one or more subjects with a specific type of cancer such as breast cancer, prostate cancer, lung cancer, colorectal cancer, or the like. In other examples, the standard represents a sample of nucleic acid obtained from one or more subjects at various progressions of cancer, such as pre-cancer, stage I, stage II, stage III, stage IV, or remission.

In another embodiment, the process of the present disclosure can be used for quantitative and qualitative detection of cfDNA.

In a further embodiment, the process of the present disclosure has an LOD of 0.4 ng/ml or 5.14 pM, and sensitivity and specificity of about 95% each for cfDNA in HEPES buffer.

In another embodiment, measuring and analyzing the conductivity can be conducted in less than 5 mins. In particular examples, measuring and analyzing the conductivity can be conducted in less than 3 mins. In further examples, measuring and analyzing the conductivity can be conducted in less than 1 minute.

In an embodiment, the biosensor and process of the present disclosure can be used for rapid, label-free screening of cancer.

The present disclosure further provides a new use of impedance spectroscopy in the detection of cancer based on the electrical conductivity of a test sample comprising cfDNA in solution.

It will now be apparent to a person of skill in the art that the present disclosure affords certain advantages over the prior art. The biosensor and methods described herein enable the direct, rapid, and label-free detection of methylated nucleic acids which are crucial markers in cancer diagnostics. This approach is sensitive enough to detect methylated nucleic acids within the clinical concentration range, negating the need for labels, amplification, surface modifications, or complex electrode geometries. A significant advantage is the reusability and cost-efficiency of the electrodes, which can be indefinitely reused with proper maintenance, only ceasing functionality when the electrodes physically wear out. Furthermore, these methods and biosensors stand out for their excellent sensitivity and specificity, each approximately 95%, ensuring a high degree of accuracy and reliability in detecting cancerous changes.

Building on these advantages, the described impedance spectroscopy methods offer a universal screening solution for a wide array of cancer types, thanks to the prevalence of aberrant methylation across all cancers. This approach contrasts with existing multi-cancer screening methods that predominantly rely on assays or DNA sequencing, presenting a faster, more cost-effective alternative. Patients will benefit from quicker, less expensive diagnostic results, while healthcare providers will be empowered to screen more frequently. Early detection is crucial for successful cancer treatment, and the increased screening frequency facilitated by this method will increase the chances of catching cancer in the early stages. Moreover, the label-free nature of these methods contributes to reduced waste production in clinical laboratories, aligning with sustainable practices in medical testing.

The biosensor and methods are further described with respect to the examples provided herein.

### EXAMPLES

### Example 1: Sample Collection and Plasma Separation

A total of 131 cancer and 40 healthy samples were taken from Maulana Azad Medical College and Rajiv Gandhi Cancer Institute and Research Centre, Delhi. The type of cancer samples collected were as follows: breast (26), thyroid (19), colorectal (4), head and neck (17), kidney (31), gall bladder (5), lymph node (4), brain (4), penis (1), prostate (7), ovary (5), pancreatic (2), cervical (1) and of unknown origin (5). From each person, 2 ml of whole blood was collected in a K3EDTA tubes via routine venous phlebotomy and the plasma was isolated from blood by double centrifugation (2000 g for 10 min followed by 16000 g for 10 min) at 4° C. The plasma layer was then transferred into cryovials and frozen immediately.

### Example 2: Extraction of Cf/ctDNA from Plasma

For cfDNA extraction, the plasma samples were first thawed at room temperature and then the cfDNA was extracted using the MagMax™ cfDNA isolation kit following the manufacturer's protocol. The cfDNA was finally eluted using an elution buffer and its amount was quantified by UV-Visible spectroscopy (Nanodrop™ Lite, Thermo Fisher Scientific). To prepare cfDNA samples, 2 ml of eluted cfDNA in the MagMax™ elution buffer was freshly spiked in 98 ml of 15 mM HEPES buffer at pH 7.4. A sample containing only 15 mM HEPES with 2% v/v elution buffer (i.e., no cfDNA) was used as reference.

### Example 3: Optimization of Frequency and Selection of Solvent for Impedance Measurement

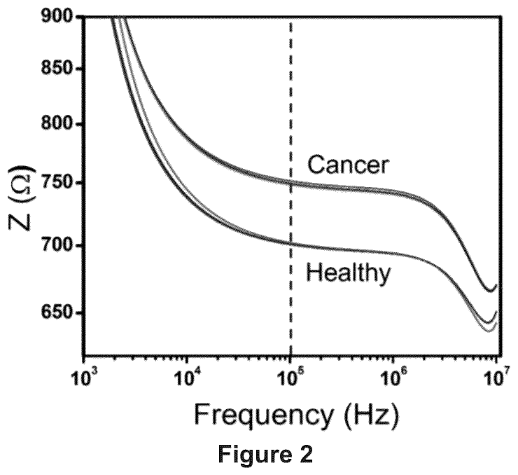

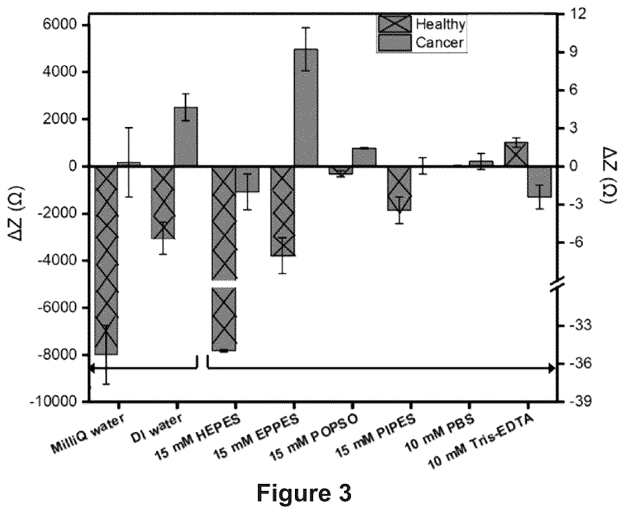

First, to obtain maximum reliable signal change between cancer and healthy samples, the operating frequency and the type of solvent used was optimized. The maximum signal difference was obtained in the 10 kHz to 1 MHz range (FIG. 2), so 0.1 MHz was fixed in all subsequent measurements. The phase angle at this frequency was much lower than 90° (data not shown) which meant that the impedance response was mostly resistive in nature. Therefore, throughout the specification, the term conductance has been used interchangeably with impedance. Further, change in impedance (ΔZ) of cfDNA samples was also determined in various liquids such as deionized (DI) water, Milli-Q™ water, 10 mM PBS, and 10 mM tris-EDTA and different zwitterionic buffers like HEPES, EPPS, POPSO and PIPES, all taken at 15 mM. Results showed that the S/N for ΔZ was highest in HEPES followed by POPSO and EPPS (FIG. 3 and Table 1).

TABLE 1

| | Conductivity | ΔZ₁ (Ω) | | ΔZ₂ (Ω) | | ΔZ = ΔZ₂ − | |
|---|---|---|---|---|---|---|---|
| Medium | (S/m) | Healthy | \|S/N\| | Cancer | \|S/N\| | ΔZ₁ (Ω) | \|S/N\| |
| 15 mM | 0.04 | −33.20 ± | 11.85 | −2.00 ± | 1.42 | −31.20 ± | 9.96 |
| HEPES | | 2.80 | | 1.40 | | 3.13 | |
| 15 mM | 0.21 | −0.59 ± | 2.56 | 1.44 ± | 28.8 | 2.03 ± | 8.45 |
| POPSO | | 0.23 | | 0.05 | | 0.24 | |
| 15 mM | 0.06 | −7.02 ± | 4.97 | 9.22 ± | 5.45 | 16.24 ± | 7.38 |
| EPPS | | 1.41 | | 1.69 | | 2.20 | |
| DI water | 4.37e−4 | (−3.05 ± | 4.48 | (2.51 ± | 4.42 | (−5.56 ± | 6.27 |
| | | 0.68) × 10³ | | 0.57) × 10³ | | 0.88) × 10³ | |
| Milli-Q ™ | 5.41e−4 | (−7.99 ± | 6.44 | (0.17 ± | 0.12 | (8.16 ± | 4.27 |
| water | | 1.24) × 10³ | | 1.46) × 10³ | | 1.91) × 10³ | |
| 10 mM tris- | 0.11 | 1.89 ± | 5.10 | −2.40 ± | 2.55 | −4.29 ± | 4.25 |
| EDTA | | 0.37 | | 0.94 | | 1.01 | |
| 15 mM | 0.33 | −3.44 ± | 3.31 | 0.04 ± | 0.07 | −3.40 ± | 2.83 |
| PIPES | | 1.04 | | 0.60 | | 1.20 | |
| 10 mM PBS | 1.75 | 0.05 ± | 2.50 | 0.39 ± | 0.61 | 0.34 ± | 0.53 |
| | | 0.02 | | 0.64 | | 0.64 | |

Table 1 above shows the comparative performance of the different media shown in FIG. 3 based on their S/N value. HEPES had the highest S/N and thus, the best performance. The S/N was lowest for PBS and intermediate for water as expected based on their conductivity values. HEPES was thus fixed as the buffer of choice for all remaining experiments. All buffers are at pH 7.4, or approximately the pH of human blood, in order to stabilize the DNA.

### Example 4: Detection of Cf/ctDNA Using Impedance Analyzer

The impedance reading was taken by placing 20 μl of the test sample on a clean pair of IDEs (100 μm×100 μm platinum on glass) enclosed in a PDMS microchamber (4 mm diameter, 1.6 mm thickness) and then covering the PDMS microchamber by a glass cover slip that was sealed using vacuum grease. Care was taken to keep the diameter and height of the microchamber fixed in order to keep the geometrical cell constant of the system uniform. The impedance reading was recorded at 100 mV over the broad frequency range of 20 Hz to 10 MHz using an impedance analyzer (E4990A, Keysight™). The reading was noted 3 min after the sample was applied to the electrode. The results were plotted as ΔZ=(Z_{cfDNA}−Z_{ref}) and reported as the mean of two experiments±1 SD.

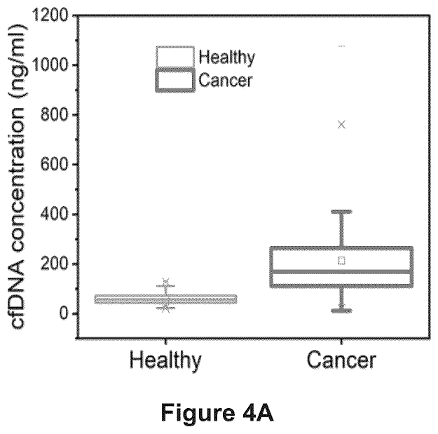

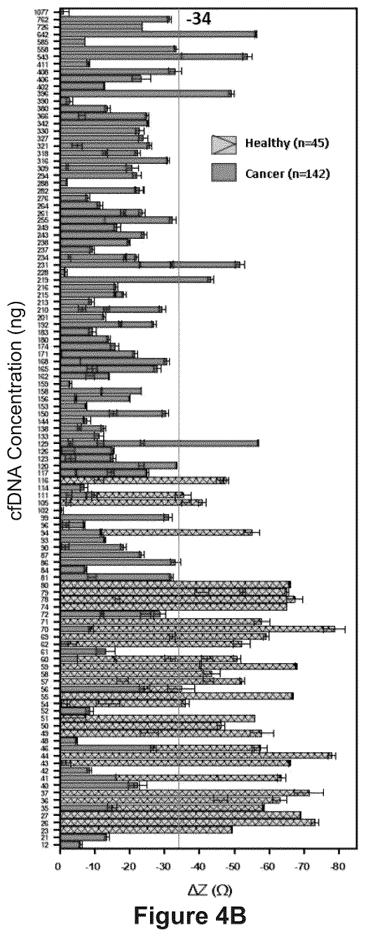

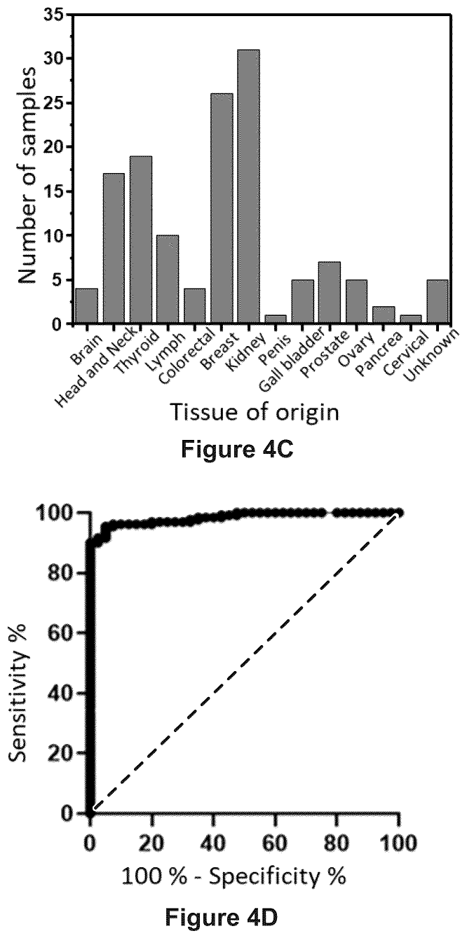

Results with clinical samples showed that the absolute value of AZ signal of cancer samples was significantly higher than that of healthy ones (FIG. 4B). After the ROC analysis, the threshold for classifying a sample as cancer-positive was set at −34 ohms where, samples with readings above this value were identified as cancer-positive and below this value as cancer-negative (or, healthy). Based on this classification, the sensitivity and specificity of the system was found to be 95% each.

### Example 5: Investigating the Reason Behind the Signal Differences

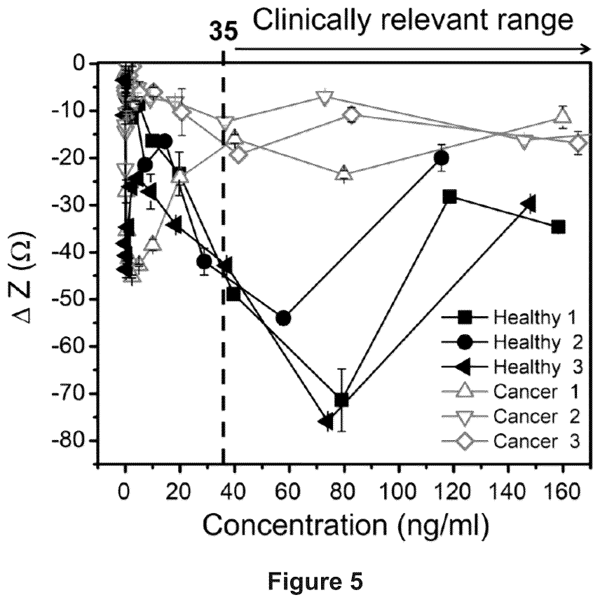

The inventors of the present disclosure found that the electrical conductivity measured in the presently proposed system is dominated by the ionic conductivity of the system. AZ response is highly non-monotonic with respect to the DNA concentration and the solvent plays an integral role in the signal quality as evidenced by the results provided in FIG. 5.

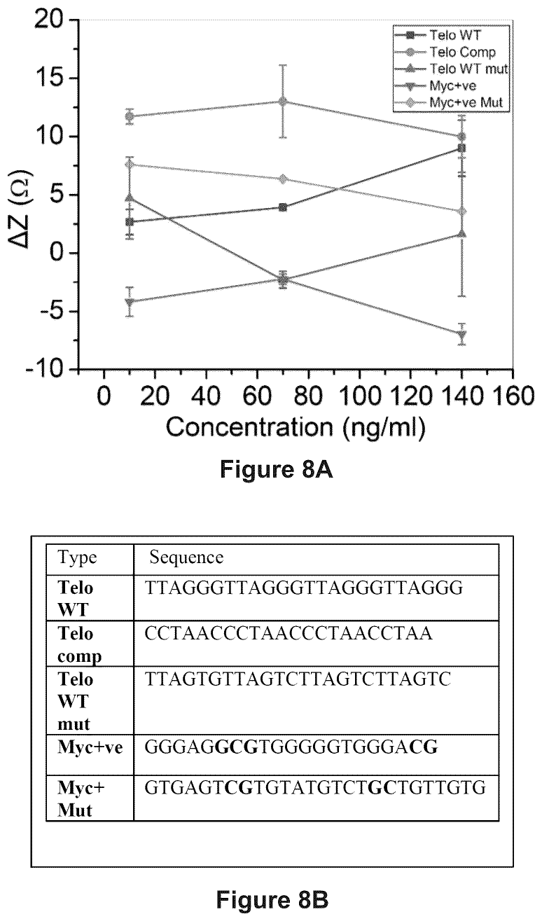

According to Marzano et al. Molecules 2019, 24, 654; G4, oligonucleotides have higher conductivity, due to high ability of base stacking and presence of trapped ions, than a normal DNA, hence a lower impedance. To check if the underlying nucleotide sequence affects the conductivity response in any way, synthetic oligonucleotide sequences with increasing GC content were tested (FIGS. 8A and 8B). FIG. 8A shows the lack of variation in impedance as a function of oligonucleotide concentration. The oligonucleotides (SEQ ID NOs: 1 to 5) had different extents of GC content as shown in the sequences of FIG. 8B. (The bolded letters in the sequences of FIG. 8B indicate methylated cytosine.) Results of this test showed no discernible differences between the various sequences, which ruled out the contribution of electronic conductivity. FIG. 5 shows that the electrical conductivities of cancer and healthy differed greatly even at the same DNA concentration which points to the underlying role of DNA morphology during impedance measurements.

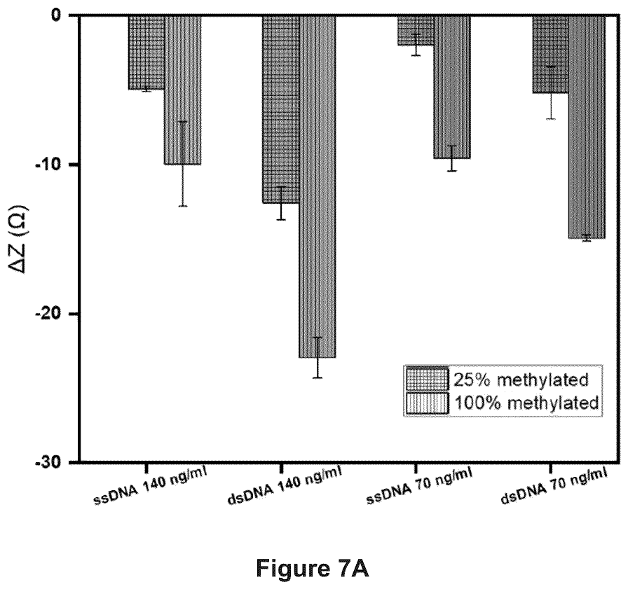

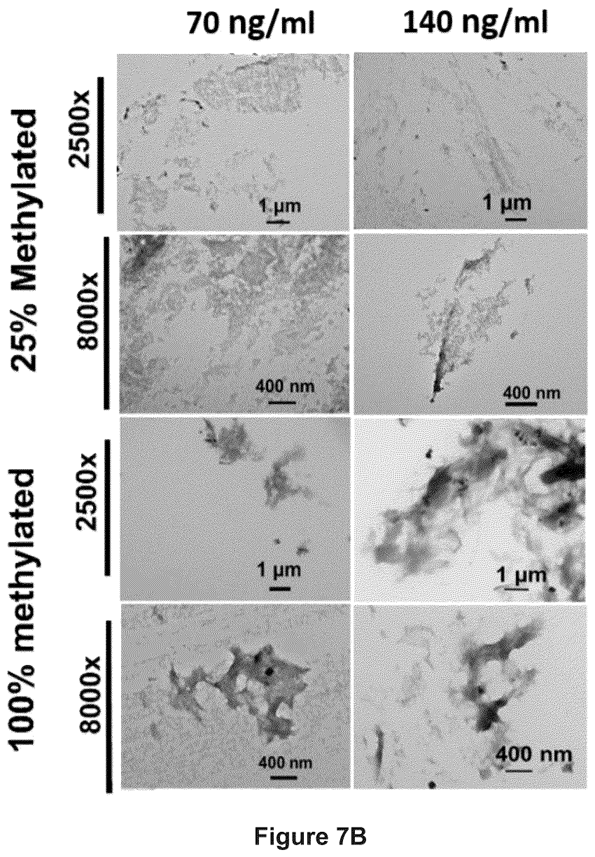

As measured in FIG. 4A, experiments were designed in which the methylation content was systematically varied along the oligonucleotide sequence corresponding to the hTERT gene promoter region. The 77-mer long sequences with their corresponding 25% and 100% methylation content were received from Sigma-Aldrich (India). In real samples, it is expected that the cfDNA derived from a healthy person will have a higher methylation percentage compared to that of a cancer patient. The present experiments were conducted with both the double-stranded (ds) and single-stranded (ss) forms of DNA as the cfDNA is known to exist in both forms. Also, the experiments were performed with two DNA concentrations (70 and 140 ng/ml) to cover the typical clinical range of cfDNA found in unhealthy tissues. The results depicted in FIG. 7A showed that dsDNA was more conductive than ssDNA which is in agreement with the literature. Also, the impedance varied non-monotonically with methylation, wherein, the 25% methylated oligos had lower signal as compared to 100% methylated oligos, irrespective of the cfDNA concentration used or the ds/ss nature of the DNA strands.

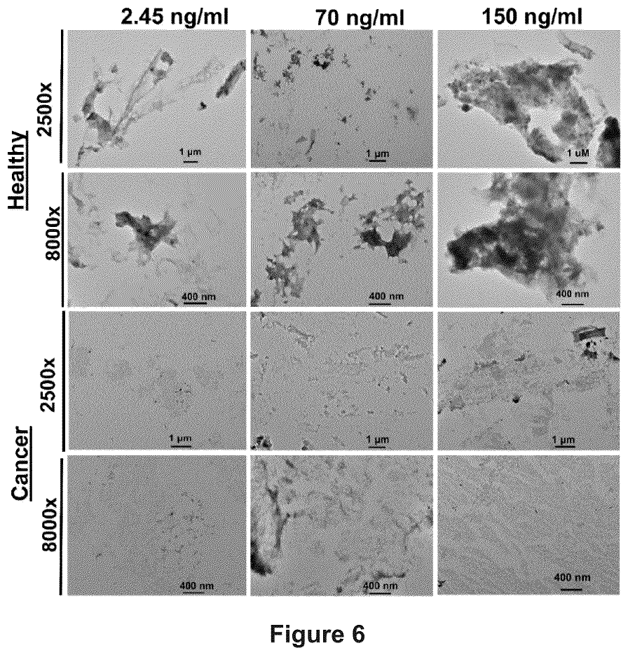

Different extents of methylation can significantly alter the DNA morphology as shown by the TEM images of the differentially methylated oligos (FIG. 7) and clinical samples (FIG. 6). FIG. 7 shows that the 25% methylated DNA is more dispersed at both 70 and 140 ng/ml concentrations. 100% methylated oligos showed higher change in impedance as compared to 25% methylated samples. As the methylation increases to 100%, even larger aggregates form that extend well beyond tens of μm in size. The same morphological behavior is seen in real samples which reveal that the healthy cfDNA with higher methylation content forms large aggregates whereas, the cancer cfDNA with less methylated content is relatively well dispersed (FIG. 6).

### Example 6: Effect of Methylation on the Impedance Signal of Single Stranded (Ss) and Double Stranded (Ds) Synthetic Oligonucleotides

The morphological differences may lead to the non-monotonic behavior observed in FIG. 7A. The reason why the 25% methylated oligos are more well-dispersed than the unmethylated oligos is because of the presence of a methyl group on the fifth position of a cytosine which tends to stabilize the DNA molecule. However, the presence of such a non-conductive functional group can disrupt the charge mobility of the counterions along the DNA backbone, leading to a dip in the ΔZ response. In the case of 100% methylated oligos, a high number of hydrophobic methyl groups in proximity can collapse the cfDNA into microsized domains surrounded by unmethylated hydrophilic regions. This lowers the total surface area per unit volume of the aggregate. In other words, the relative contribution of the counterionic charges to the overall conductivity is reduced compared to the core mass of aggregate that does not conduct. This leads to the difference in the magnitude of signal achieved from 100% and 25% methylated oligos. This aggregation behavior persists across the different cfDNA concentrations in the clinical range (FIG. 6) which implies that the presently proposed method is applicable irrespective of the cfDNA concentration obtained from blood plasma. Also, the effect of methylation on impedance follows matching trends in FIGS. 4B and 7A, which confirms that the present method gives true information of epigenomic signatures.

### Example 7: Comparative Analysis with Other Available Methods

The present approach was compared and analysed with various known techniques for various parameters including LOD, detection time, type of receptor, type of biomarker etc. Results are illustrated in Table 2 below:

TABLE 2

| | | | Signal | | | | | |
|---|---|---|---|---|---|---|---|---|
| Detection | | | amplification | Linear | | | | |
| method | Target | Receptor | method | range | LOD | Time | | Characteristics |
| Electrochemical | ctDNA | Probe | Amplification | 0.1 to | 1 aM | — | | Label free, enzyme |
| (IS) | | DNA | free | 100 fM | | | | free, use of |
| | | | | | | | | MoS₂/graphene |
| | | | | | | | | composites |
| Electrochemical | DNA | Receptorless | NP | — | 25-30 | — | | Reusable chips, |
| (IS) | PCR | | labeling | | DNA | | | dielectrophoretic |
| | products | | | | copies | | | concentration of |
| | | | | | | | | analyte |
| Electrochemical | cfNA | PNA | Amplification | 10.27 | 10.27 fM | 15 | min | Multiplexing, highly |
| (DPV) | (KRAS | capture | free | fM-1.03 | | | | specific |
| | and | probe | | nM | | | | |
| | BRAF | | | | | | | |
| | mutation) | | | | | | | |
| Electrochemical | Methylated | Direct | Amplification | — | — | 10 | min | Genomic distribution |
| (DPV) | genomic | genomic | free | | | | | of methylcytosines |
| | DNA | DNA | | | | | | on the |
| | | adsorption | | | | | | physicochemical |
| | | | | | | | | properties of DNA |
| Electrochemical | cfDNA | Triple- | Target | 10 aM - | 2.4 aM | | | |
| (DPV) | (KRAS | helix | recycling + | 1 pM | | | | |
| | G12DM) | molecular | branched | | | | | |
| | | switch | TdT | | | | | |
| Electrochemical | ctDNA | DNA- | MB | 200 aM - | 5 fM | 20 | min | Preconcentration of |
| (SWV) | (101 | Au@M | tagged | 20 nM | | | | ctDNA on sensing |
| | nucleotides, | NPs | on | | | | | interface |
| | NSCLC) | | electrode | | | | | 20 |
| | | | surface | | | | | |
| | | | and | | | | | |
| | | | Au@MN | | | | | |
| | | | Ps | | | | | |
| Electrochemical | ctDNA | PNA | AuNPs | 50- | 10 fM | 30 | min | |
| (SWV) | (PIK3CA | and | and lead | 10000 | | | | |
| | Mutation and | anti-5- | phosphate | fM | | | | |
| | methylation) | mC | apoferritin | | | | | |
| Electrochemical | Methylated | Tetrahedral | AuNP | 1 aM - 1 | 1 aM | 6 | h | 22 |
| (chronoamperometry) | oligos | DNA | deposition | pM | | | | |
| | | nanostructure | on | | | | | |
| | | probe | electrode, | | | | | |
| | | | HCR | | | | | |
| | | | and HRP | | | | | |
| Electrical (IS) | DNA | Receptor | Amplification | — | 148 bp | — | | Label free, reusable |
| | PCR | less | free | | | | | chips, direct |
| | products | | | | | | | impedance of |
| | | | | | | | | measurement DNA |
| | | | | | | | | 23 |
| Impedance | cfDNA | Receptorless, | Amplification | — | 0.4 | 3 | min | Effect of |
| based cfDNA | | direct | free | | ng/ml or, | | | methylation pattern |
| detection using | | cfDNA | | | 5.14 pM | | | of cfDNA on |
| Zwitterionic | | detection | | | | | | electrophysiological |
| buffer | | in bulk | | | | | | properties in HEPES |
| | | | | | | | | buffer |
| | | | | | | | | Our work |

IS: Impedance Spectroscopy;
DPV: Differential pulse voltammetry;
SWV: Square-wave voltammetry;
PNA; Peptide nucleic acids;
AuNP: gold nanoparticles;
anti-5-mC: Monoclonal anti-5-methylcytosine antibody;
PCR: Polymerase chain reaction;
ctDNA: circulating tumor DNA;
DNA-Au@MNPs: DNA modified gold-coated magnetic NPs;
HCR: hybridization chain reaction;
HRP: Horseradish peroxidase;
TdT: terminal deoxynucleotidyl transferase;
MB: methylene blue;
NSCLS; Non-small cell lung cancer

TABLE 3
Sequence of synthetic oligomer used
in methylation experiments

| | | |
|---|---|---|
| 25% | SEQ ID | AACCCGAGGACGCATTGCTCCCTGGACG |
| methylated | NO: 6 | GGCACGCGGGACCTCCCGGAGTGCCTCC |
| | | CTGCAACACTTCCCCGC |
| 100% | SEQ ID | AACCCGAGGACGCATTGCTCCCTG |
| methylated | NO: 7 | GACGGGCACGCGGGACCTCCCGGA |
| | | GTGCCTCCCTGCAACACTTCCCCGC |

In Table 3, the underling indicates 5-methylcytosine.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

We claim:

1. A biosensor (100) for analyzing a test sample comprising DNA, the biosensor comprising:
a microchamber (101) configured to hold said test sample suspended in a zwitterionic buffer, wherein said microchamber encloses a plurality of electrodes (102); and
an impedance analyzer (105) operably connected to said microchamber and a programmable controller along with a digital processing unit (104) to receive information from the plurality of electrodes;
wherein impedance signals are measured by placing said test sample on the plurality of electrodes.

2. The biosensor of claim 1, wherein the microchamber comprises a material selected from polydimethylsiloxane, polylactic acid, polylactic-co-glycolic acid, polyether ether ketone, silicone, nitrile, polyurethane, soft vinyl chloride resin, polypropylene, polyamide, polyethylene, polycarbonate, acrylonitrile butadiene styrene (ABS) resin, polystyrene, and poly(methyl methacrylate).

3. The biosensor of claim 1 for use in rapid, label-free, and amplification-free screening of cancer.

4. A method for liquid biopsy, using a zwitterionic buffer and impedance spectroscopy, comprising the steps of:
a. collecting a biological sample and extracting a cfDNA fraction from said biological sample;
b. suspending said cfDNA fraction in the zwitterionic buffer to prepare a test sample;
c. loading said test sample over a plurality of electrodes enclosed inside a microchamber;
d. measuring the electrical conductivity of said test sample using an impedance analyzer; and
e. analyzing a difference between the electrical conductivity of said test sample and the electrical conductivity of a control sample.

5. The method of claim 4, wherein the biological sample comprises a biological fluid selected from whole blood, plasma, platelets, saliva, white blood cells, serum, and urine.

6. The method of claim 4, wherein the biological sample comprises plasma, the method further comprising:
separating the plasma from whole blood via double centrifugation, the double centrifugation comprising a first cycle at 1500 g to 2500 g and preferably 2000 g for 7-12 minutes and preferably 10 minutes, and a second cycle at 15000-17000 g and preferably 16000 g for 7-12 minutes and preferably 10 minutes at 4° C.

7. The method of claim 4, wherein the zwitterionic buffer is selected from (4-(2-hydroxyethyl)-1-piperazineethanesulfonic acid), piperazine-1,4-bis(2-hydroxypropanesulfonic acid), and 4-(2-Hydroxyethyl)-1-piperazinepropanesulfonic acid.

8. The method of claim 4, wherein said method can be used for quantitative and qualitative detection of cfDNA.

9. The method of claim 7, wherein the zwitterionic buffer comprises (4-(2-hydroxyethyl)-1-piperazineethanesulfonic acid) and wherein said method has a limit of detection of 0.4 ng/ml or 5.14 pM and a sensitivity and specificity each of about 95%.

10. The method of claim 4, wherein measuring and analyzing the electrical conductivity is performed in less than 5 minutes and preferably less than 1 minute.

11. Use of impedance spectroscopy in the detection of cancer based on the electrical conductivity of a test sample comprising cfDNA in solution.

12. A label-free method of detecting cfDNA using impedance spectroscopy, comprising the steps of:
a. collecting a biological sample and extracting a cfDNA fraction from said biological sample;
b. suspending said cfDNA fraction in a liquid to prepare a test sample;
c. loading said test sample over a plurality of electrodes enclosed inside a microchamber;
d. measuring the electrical conductivity of said test sample using an impedance analyzer; and
e. analyzing a difference between the electrical conductivity of said test sample and the electrical conductivity of a control sample.

13. The method of claim 12 wherein the liquid is selected from a zwitterionic buffer, deionized water, and purified water.

14. The method of claim 12, wherein the biological sample comprises a biological fluid selected from whole blood, plasma, platelets, saliva, white blood cells, serum, and urine.

15. The method as claimed in claim 12, wherein the biological sample comprises plasma, the method further comprising:
separating the plasma from blood via double centrifugation, the double centrifugation comprising a first cycle at 1500 g to 2500 g and preferably 2000 g for 7-12 minutes and preferably 10 minutes, and a second cycle at 15000-17000 g and preferably 16000 g for 7-12 minutes and preferably 10 minutes at 4° C.

16. The method of claim 13, wherein the liquid comprises a zwitterionic buffer selected from (4-(2-hydroxyethyl)-1-piperazineethanesulfonic acid), piperazine-1,4-bis(2-hydroxypropanesulfonic acid), and 4-(2-Hydroxyethyl)-1-piperazinepropanesulfonic acid.

17. The method of claim 12, wherein said method can be used for quantitative and qualitative detection of cfDNA.

18. The method of claim 12, wherein measuring and analyzing the electrical conductivity is performed in less than 5 minutes and preferably less than 1 minute.

19. A method of detecting DNA using impedance spectroscopy, comprising the steps of:
a. collecting a biological sample and extracting a DNA fraction from said biological sample;
b. suspending said DNA fraction in a zwitterionic buffer to prepare a test sample;
c. loading said test sample over a plurality of electrodes enclosed inside a microchamber;
d. measuring the electrical conductivity of said test sample using an impedance analyzer; and
e. analyzing a difference between the electrical conductivity of said test sample and the electrical conductivity of a control sample.

20. The method of claim 19, wherein the DNA fraction comprises at least one of genomic DNA (gDNA), cell free DNA (cfDNA), mitochondrial DNA (mtDNA), and complementary DNA (cDNA).

21. The method of claim 19, wherein the biological sample comprises a biological fluid selected from whole blood, plasma, platelets, saliva, white blood cells, serum, and urine.

22. The method as claimed in claim 19, wherein the biological sample comprises whole blood, the method further comprising separating a buffy coat layer from the whole blood.

23. The method of claim 19, wherein the zwitterionic buffer is selected from (4-(2-hydroxyethyl)-1-piperazineethanesulfonic acid), piperazine-1,4-bis(2-hydroxypropanesulfonic acid), and 4-(2-Hydroxyethyl)-1-piperazinepropanesulfonic acid.

24. The method of claim 19, wherein said method can be used for quantitative and qualitative detection of DNA.

25. The method of claim 19, wherein measuring and analyzing the electrical conductivity is performed in less than 5 minutes and preferably less than 1 minute.


SEQUENCE LISTING (7 sequences)

SEQ ID NO: 1 - DNA, 24 bp, Synthetic Construct
  source 1..24: mol_type = other DNA; note = Oligonucleotide used as test sample
ttagggttagggttagggttaggg

SEQ ID NO: 2 - DNA, 22 bp, Synthetic Construct
  source 1..22: mol_type = other DNA; note = Oligonucleotide used as test sample
cctaaccctaaccctaacctaa

SEQ ID NO: 3 - DNA, 24 bp, Synthetic Construct
  source 1..24: mol_type = other DNA; note = Oligonucleotide used as test sample
ttagtgttagtcttagtcttagtc

SEQ ID NO: 4 - DNA, 21 bp, Synthetic Construct
  source 1..21: mol_type = other DNA; note = Oligonucleotide used as test sample
gggaggcgtgggggtgggacg

SEQ ID NO: 5 - DNA, 26 bp, Synthetic Construct
  source 1..26: mol_type = other DNA; note = Oligonucleotide used as test sample
gtgagtcgtgtatgtctgctgttgtg

SEQ ID NO: 6 - DNA, 73 bp, Synthetic Construct
  modified_base 11: mod_base = OTHER; note = 5-methylcytosine
  modified_base 31: mod_base = OTHER; note = 5-methylcytosine
  modified_base 60: mod_base = OTHER; note = 5-methylcytosine
  source 1..73: mol_type = other DNA; note = Oligonucleotide used as test sample
aacccgaggacgcattgctccctggacgggcacgcgggacctcccggagtgcctccctgcaacacttccccgc

SEQ ID NO: 7 - DNA, 73 bp, Synthetic Construct; identical to SEQ ID NO: 6
  modified_base 5: mod_base = OTHER; note = 5-methylcytosine
  modified_base 11: mod_base = OTHER; note = 5-methylcytosine
  modified_base 13: mod_base = OTHER; note = 5-methylcytosine
  modified_base 18: mod_base = OTHER; note = 5-methylcytosine
  modified_base 27: mod_base = OTHER; note = 5-methylcytosine
  modified_base 31: mod_base = OTHER; note = 5-methylcytosine
  modified_base 33: mod_base = OTHER; note = 5-methylcytosine
  modified_base 35: mod_base = OTHER; note = 5-methylcytosine
  modified_base 45: mod_base = OTHER; note = 5-methylcytosine
  modified_base 52: mod_base = OTHER; note = 5-methylcytosine
  modified_base 60: mod_base = OTHER; note = 5-methylcytosine
  modified_base 71: mod_base = OTHER; note = 5-methylcytosine
  modified_base 73: mod_base = OTHER; note = 5-methylcytosine
  source 1..73: mol_type = other DNA; note = Oligonucleotide used as test sample